(19) United States
(12) Patent Application Publication
(10) Pub. No.: US 2023/0149737 A1
(43) Pub. Date: May 18, 2023

(54) METHOD AND APPARATUS FOR TREATMENT OF PULMONARY INFLAMMATION

(71) Applicant: KAST Allen George, Conroe, TX (US)

(72) Inventors: George Allan KAST, Conroe, TX (US); Rehab AMIN, Conroe, TX (US)

(21) Appl. No.: 17/923,197

(22) PCT Filed: May 4, 2021
(86) PCT No.: PCT/US2021/030727

§ 371 Date: Nov. 3, 2022

Related U.S. Application Data
(60) Provisional application No. 63/020,300, filed on May 5, 2020.

(51) Int. Cl. A61N 5/067; A61N 5/06

(52) U.S. Cl. CPC A61N 5/067; A61N 5/0616; A61N 2005/0645; A61N 2005/0651

(57) ABSTRACT

The present disclosure includes a method of using energy, particularly photonic energy, to promote the anti-inflammatory effect and the process of biological alteration to restore the normal function of the lungs of infected patients without the need of invasive means or use of chemical compositions. The disclosed apparatus and process disclosed herein treats respiratory and other viral infections as well as inflammation through the application of photonic energy into the human body. To accomplish this goal in an efficient and cost effective manner, a wearable unit is disclosed herein that contains a photonic energy source that can be coupled or worn on a human body in order to transmit photonic energy into a selected area of the body.

### CROSS REFERENCE

This application claims the benefit of U.S. Provisional Pat. Application No. 63/020,300 filed May 5, 2020, the entirety of which is hereby incorporated by reference.

### BACKGROUND

### Field

The instant invention relates to the health care industry and, more particularly, to an apparatus and related methods for treatment of pulmonary inflammation, such as is caused by respiratory viruses, and respiratory disorders associated with infection.

### Related Art

Considerable global efforts are being undertaken to address the COVID-19 pandemic and to develop methods to treat and stop the spread of this and other virus strains that are known and will likely develop in the future. This humanitarian crisis needs a treatment methodology that is immediately available and that can be implemented in the United States and globally on a mass scale to heal and restore lung functions and save lives. Aspects of this disclosure are related to a method of treatment that meets these objectives.

### SUMMARY

According to one aspect of the disclosure, a method of treating pulmonary inflammation using a wearable garment, includes: placing a wearable garment in contact with a torso of a human body having the pulmonary inflammation, the wearable garment including an irradiation source having a photon energy output primarily emitted in a spectral wavelength range of between 600-1600 nm; and directing the photon energy output of the wearable garment primarily to the torso of the human body.

According to another aspect, the method includes directing the photon energy output of the wearable garment primarily to the torso of the human body for a treatment period of between 10 and 40 minutes. According to another aspect, the method includes directing the photon energy output of the wearable garment primarily to the torso of the human body for a treatment period of at least 10 minutes. According to another aspect, the photon energy output is emitted primarily in a spectral wavelength range between 800-1000 nm. According to another aspect, the photon energy output is emitted primarily in a spectral wavelength range between 830-980 nm. According to another aspect, the photon energy output is emitted primarily in a spectral wavelength range between 630-650 nm. According to another aspect, the photon energy output is emitted primarily at a spectral wavelength of about 630 nm. According to another aspect, the photon energy output is emitted primarily at a spectral wavelength of about 680 nm. According to another aspect, the photon energy output is emitted primarily at a spectral wavelength of about 810 nm. According to another aspect, the photon energy output is emitted primarily at a spectral wavelength of about 830 nm. According to another aspect, the photon energy output is emitted primarily at a spectral wavelength of about 904 nm. According to another aspect, the irradiation source comprises a coherent light sources. According to another aspect the coherent light sources is a laser. According to another aspect, the irradiation source comprises a non-coherent light sources. According to another aspect the non-coherent light sources is a light emitting diodes (LED). According to another aspect the non-coherent light sources is a filtered lamp. According to another aspect, the irradiation source is one of an array of irradiation sources on the wearable garment. According to another aspect, the photon energy output is emitted in a pulsating pattern. According to another aspect, the photon energy output emitted sweeps over a range of spectral wavelengths. According to another aspect, the range of spectral wavelengths is between 800-1000 nm. According to another aspect, the wearable garment comprises an adjustable strap and a front piece including a first array of irradiation sources configured to primarily direct the photon energy output from the first array toward a chest region of the human body, and the strap is positioned circumferentially around the torso of the human body. According to another aspect, the adjustable strap further includes a second array of irradiation sources configured to primarily direct the photon energy output from the second array toward the chest region of the human body. According to another aspect, the wearable garment further includes a back piece including a second array of irradiation sources configured to primarily direct the photon energy output from the second array toward a back region of the human body opposite the chest region. According to another aspect, the wearable garment comprises a vest including a front piece and a back piece configured to primarily direct the photon energy output to the torso of the human body. According to another aspect, the irradiation source includes a lens configured to protrude from an inner surface of the wearable garment such that the lens contacts an outer layer of skin of the torso of the human body. According to another aspect, the pulmonary inflammation is caused by a viral infection. According to another aspect, the viral infection is COVID-19. According to another aspect, the method includes directing the photon energy output of the wearable garment primarily to a chest region, a back region opposite a chest region, or both a chest region and a back region opposite a chest region of the human body. According to another aspect, the wearable garment is placed in contact with skin of the human body. According to another aspect, the photon energy output is directed to at least 20% of a circumference around the torso. According to another aspect, the photon energy output is directed to at least 50% of a circumference around the torso. According to another aspect, the photon energy output is directed to at least 75% of a circumference around the torso. According to another aspect, the photon energy output is directed to 100% of a circumference around the torso.

According to a second aspect of the disclosure, an apparatus for treatment of COVID-19 respiratory virus, includes: a wearable garment configured to at least partially cover a chest region of a human body. The wearable garment includes: a front piece having a first array of irradiation sources. The first array of irradiation sources are configured to direct a photon energy output primarily in a spectral wavelength range between 600-1600 nm at the chest region when the wearable garment is being worn.

According to another aspect, the first array of irradiation sources comprises LEDs. According to another aspect, further including a battery configured to power the first array of irradiation sources. According to another aspect, the photon energy output is emitted primarily in a spectral wavelength range between 800-1000 nm. According to another aspect, the photon energy output is emitted primarily in a spectral wavelength range between 830-980 nm. According to another aspect, the photon energy output is emitted primarily in a spectral wavelength range between 630-650 nm. According to another aspect, the photon energy output is emitted primarily at a spectral wavelength of about 830 nm. According to another aspect, the photon energy output is emitted primarily at a spectral wavelength of about 904 nm. According to another aspect, the first array of irradiation sources comprises coherent light sources. According to another aspect, the first array of irradiation sources is configured to emit photon energy in a pulsating pattern. According to another aspect, the first array of irradiation sources is configured to emit photon energy in a sweep over a range of spectral wavelengths. According to another aspect, the range of spectral wavelengths is between 800-1000 nm. According to another aspect, the wearable garment further comprises: an adjustable strap; a mechanical fastener to couple the front piece with the chest region of the human body; and a second array of irradiation sources configured to primarily direct the photon energy output in the chest region of the human body. According to another aspect, the second array of irradiation sources is on the adjustable strap. According to another aspect, the adjustable strap includes a widened portion forming a back piece, the back piece including the second array of irradiation sources. According to another aspect, the front piece of the wearable garment has a width that extends above a chest line of the human body. According to another aspect, the wearable garment comprises a vest including the front piece and a back piece. According to another aspect, the irradiation source of the first array of irradiation sources include a lenses configured to protrude from an inner surface of the wearable garment such that the lenses are configured to contact an outer layer of skin of the chest region of the human body.

The foregoing summary is illustrative only and is not intended to be limiting. Other aspects, features, and advantages of the systems, devices, and methods and/or other subject matter described in this application will become apparent in the teachings set forth below. The summary is provided to introduce a selection of some of the concepts of this disclosure. The summary is not intended to identify key or essential features of any subject matter described herein.

### BRIEF DESCRIPTION OF THE DRAWINGS

Various examples are depicted in the accompanying drawings for illustrative purposes, and should in no way be interpreted as limiting the scope of the examples. Various features of different disclosed examples can be combined to form additional examples, which are part of this disclosure.

FIG. 1 illustrates the effects of phototherapy on a lung region of a human body at a cellular level;

FIGS. 2A-B illustrate the penetration of photon emitted from a light source that contacts an epidermal layer of a human body relative to a non-contacting light source;

FIGS. 3A-D show a wearable phototherapy garment;

FIGS. 4A-C show the wearable phototherapy garment of FIG. 3 on a torso region of a human body;

FIGS. 5A-B show another embodiment of a wearable phototherapy garment;

FIGS. 6A-C show the wearable phototherapy garment of FIG. 5 on a torso region of a human body;

FIGS. 7A-B show another embodiment of a wearable phototherapy garment;

FIGS. 8A-B show the wearable phototherapy garment of FIG. 3 on a torso region of a human body;

FIGS. 9A-B show another embodiment of a wearable phototherapy garment.

### DETAILED DESCRIPTION

The various features and advantages of the systems, devices, and methods of the technology described herein will become more fully apparent from the following description of the examples illustrated in the figures. These examples are intended to illustrate the principles of this disclosure, and this disclosure should not be limited to merely the illustrated examples. The features of the illustrated examples can be modified, combined, removed, and/or substituted as will be apparent to those of ordinary skill in the art upon consideration of the principles disclosed herein.

### Overview of Treatment Methodology for Respiratory Disorders

The field of phototherapy is based on the observation that certain wavelengths of photon light energy have been shown to penetrate deep into human body tissues for therapeutic effects. In particular, the Red and Near Infrared (NIR) light in the 600 thru 1600 nanometer range has shown to penetrate into the hypodermis layer of the body. The present disclosure includes insights and improvements to methods and applicators for the treatment of respiratory/pulmonary inflammation, including inflammation associated with respiratory/pulmonary viruses, disorders associated with viral infection(particularly, coronavirus infections such as COVID-19), asthma, and Chronic obstructive pulmonary disease (COPD).

In the present disclosure, the irradiation sources can be placed in various locations around the human body or may be localized to treat particular areas. By way of illustrative example, in one or more embodiments, the system may be configured to target the human body lung area to treat pulmonary inflammation, such as that associated with a viral infection, or to treat the complications associated with viral infections that affect the respiratory system. One example of this type of virus is COVID- 19. In treating COVID-19, the system may be configured to target the lungs and other components of the respiratory system. Likewise, it may target other areas as well.

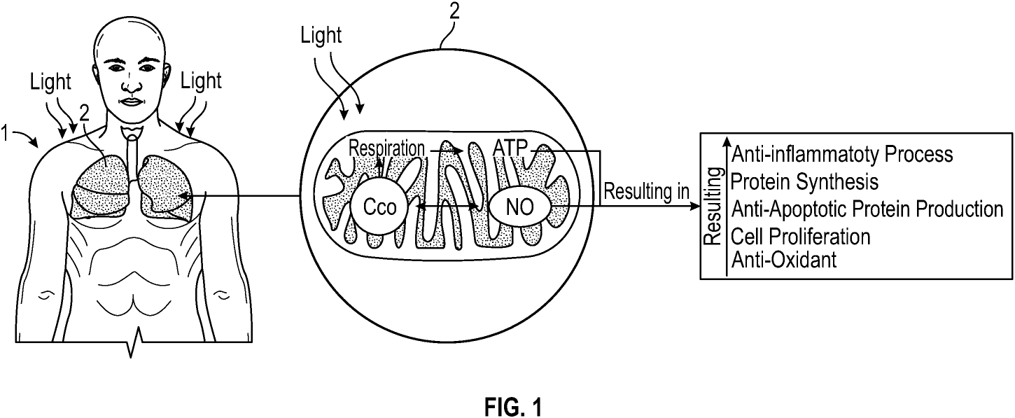

While not being bound by any particular theory, as shown in FIG. 1, light applied to a torso region 2 of a human body 1 can be absorbed by lung cells. The light can be absorbed by the photosensitive mitochondrial chromophores of the lung cells and, particularly, the Cytochrome C Oxidase (CcO) enzymes. This absorption has been shown to increase production of adenosine triphosphate (ATP) and photodissociation of inhibitory Nitric Oxide (NO) from CcO leading to increased protein synthesis, anti-apoptotic protein production, and the enhancement of cell proliferation, survival, tissue repair and regeneration. Some of the benefits of this approach include the ability to reduce lung inflammation by reducing the number of inflammatory cytokines and mast cell degranulation in a non-intrusive manner.

In some embodiments, light may be applied to a torso region using a wearable garment. As used herein, “wearable garment” refers to an apparatus containing at least one flexible portion and that can be secured to a human body such that it stays in close proximity to (including but not limited to in contact with) the body. The wearable garment, as described herein, can include, but is not limited to a strap, vest, shirt, jacket, sleeve, gown, or one or more flexible or rigid pads attachable to a human body 1. In some embodiments, the wearable garment is secured so that at least one portion stays in contact with the human body (e.g., in contact with the skin of the human body). In some embodiments, the wearable garment is secured so that the human body 1 may move about while the wearable garment stays in close proximity to the human body 1. The wearable garment (as more fully described below) can include an array of light sources that primarily direct light energy to the torso region 2 of a human body 1. The energy may be generated by coherent light sources such as lasers, noncoherent light sources consisting of filtered lamps or LEDs, or a combination thereof. A significant benefit of this method is that it can be administered easily and in large numbers and it can be applied in many different settings such as hospitals, doctor’s offices, health clinics, chiropractor’s offices, medspas, and many other venues including as a home treatment.

The array can include light sources arranged in a grid or other pattern. The emitted light from the light sources of the array can be uniform or differentiated. If uniform, the light emitted can having the same wavelengths or range of wavelengths. If different, certain light sources can emit light at one wavelengths or range of wavelengths, while other light sources emit light at different wavelengths or ranges of wavelengths. The wavelengths or ranges of wavelengths can be selected for therapeutic effect.

The energy used for this method can be generated in one of a number of manners, and each may have particular benefits or drawbacks based on the intended applications. Accordingly, systems and methods disclosed herein may include coherent light sources such as lasers, non-coherent light sources consisting of filtered lamps or light emitting diodes (commonly referred to as “LED’s”), or a combination thereof.

The treatment methodologies can include exposure to the light sources of the array of the wearable garment for a period of time and/or cycles of usage. The user will apply the energy source for a pre-determined amount of time of exposure, then turn off the energy source and remove the wearable. Optionally, a controller may be used to select the treatment (e.g., wavelength(s), length of usage, sweeping, pulsating, etc.). Preferably, the particular wearable garment may be fitted with specific energy emitting sources for particular ranges. Alternatively, the wearable garment can include a control that includes a pre-designated treatment regime or a selection of pre-designated treatment regimes. After completing a usage block, the wearable garment can turn off and/or provide an alert to the user.

The total energy delivered to a patient during a treatment session should be calculated to deliver the desired therapeutic effect. For example, treatment of early-stage pulmonary inflammation (e.g., due to early-stage COVID-19) may require less total light dosage per session that advanced-stage pulmonary inflammation. Non-limiting doses used for the methods described herein include the following:

| Dosage per Session (J/cm²) | Treatment Type |
|---|---|
| 4-6 | Early |
| 6-10 | Moderate |
| 10-20 | Severe |

Other dose ranges for the uses described herein include 1-20 J/cm², 2-15 J/cm², 4-10 J/cm², and 2-5 J/cm².

The treatment duration can vary depending on the total intensity of the light sources of the wearable garment. According to one example, the wearable garment can be worn and used for a period of about 5-60 minutes, 10-50 minutes, 10-45 minutes, 10-30 minutes, 30-60 minutes, or 45-60 minutes. According to another example, the wearable garment can be worn and used for a cumulative period of between about 1-10 hours over several sessions. An example schedule for the treatment of respiratory disease, and particularly COVID-19 is provided below.

| Minutes per Session | Sessions |
|---|---|
| 20 | 3-8 |
| 40 | 3-8 |
| 60 | 3-10 |

In certain examples, the power output capacity of the light sources of the wearable device can be within the range of about 1-100 mW/cm², 10-80 mW/cm², 5-30 mW/cm², 5-15 mW/cm², 30-60 mW/cm², or about 10 mW/cm². In certain examples, the light sources, in total, can deliver about 12 W of power to the torso region. Devices delivering lesser or higher output intensities can also be used and the dosage and/or session lengths can be varied according for the desired therapeutic effect. Devices using fewer or greater numbers of lights sources can be used depending on the output capacity of the individual light sources (e.g., LEDs).

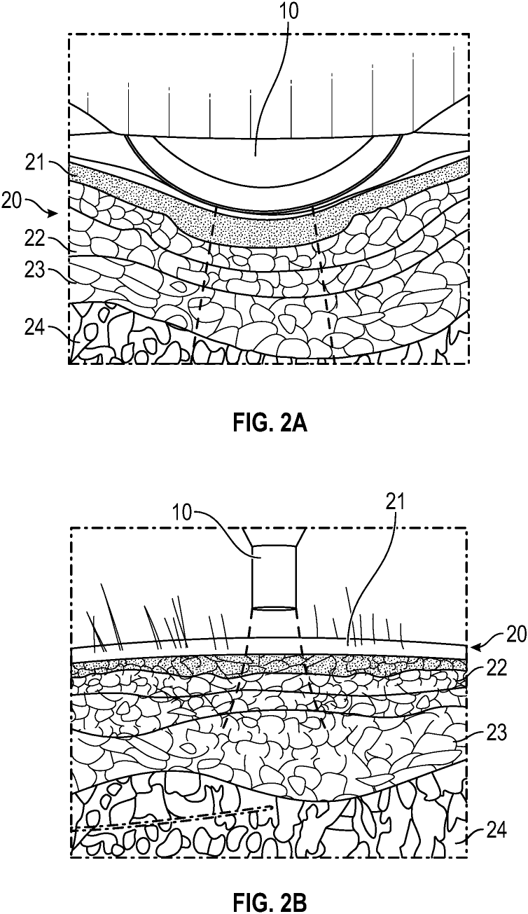

The power delivered by the lights sources to the skin is affected by the distance between the two. A closer proximity leads to more power being delivered and less proximity lead to greater losses in delivered power. As illustrated in FIG. 2A, contact between a lens of a light source 10 and the skin 20 of a user, can increase the intensity and/or penetration depth of photons entering the skin 20 relative to the spaced light source 10 shown in FIG. 2B. By allowing the lens to contact the epidermis 21, photons can more easily pass through the dermis 22, the hypodermis 23, and through muscle 24. Accordingly, the lenses of the light sources 10 of the wearable garment can protrude outwardly from an inner surface of the garment. The protruding lenses can facilitate contact between the skin of a patient using the wearable garment.

During treatment, a user will place the wearable garment onto its body (e.g., in direct contact with skin of the torso 2) and will activate the energy emitting sources to emit light. Photon energy from the light sources of the array are operationally configured into the wearable garment such that the photon energy emitted therefrom is directed into the cavity formed within the wearable garment. Ideally, the light sources are placed in a manner to focus and direct energy broadly so as to cover the entirety of the human’s exposure area (e.g., torso), but they may be focused for specific applications. Moreover, the energy sources may be positioned to minimize discomfort to the patient.

The wavelengths of the emitted light can be in the 600 thru 1600 nm range. In certain applications the wavelengths can range from 800-1200 nm. In certain applications the wavelengths can range from 800-1000 nm. Particularly for addressing respiratory infections from viral sources, the wavelengths can range primarily in a spectral wavelength range between 620 nm - 640 nm, 670 nm - 690 nm, 800 nm - 820 nm, 820 nm - 840 nm, and/or 895 nm - 915 nm. Particularly for addressing respiratory infections from viral sources, the wavelengths can be primarily at a spectral wavelength about 630 nm, 680 nm, 810 nm, 830 nm and/or 904 nm (or any combination thereof).

According to one implementation for addressing viral respiratory infections, and particularly COVID-19, an array including light sources at about 830 nm and 904 nm has been shown to be clinically effective at reducing symptoms. According to one implementation for addressing viral respiratory infections, and particularly COVID-19, an array including light sources only at about 830 nm and 904 nm has been shown to be clinically effective at reducing symptoms. According to one implementation for addressing viral respiratory infections, and particularly COVID-19, an array including light sources between 830-980 nm has been shown to be clinically effective at reducing symptoms. According to one implementations for addressing viral respiratory infections, and particularly COVID-19, an array including light sources only between 830-980 nm has been shown to be clinically effective at reducing symptoms.

In various embodiments, the light provided by the array of light sources may be at one, two, three, or more different wavelengths or different ranges of wavelengths. Where more than one wavelength or range of wavelengths are provided, different light sources for the different wavelengths or ranges of wavelengths may be utilized. For example, the wearable garment may have multiple arrays of light sources, where each array produces light at a distinct wavelength or range of wavelengths. Alternatively, each array of light sources may include light sources that emit light at different wavelengths or ranges of wavelengths. For example, in some embodiments, the array of light sources include alternating light sources of different wavelengths or ranges of wavelengths.

The light sources of the arrays can be varied in intensity. While not being bound by any particular theory, pulsing the light intensity has been shown to increase stimulation of cells leading to increased production of ATP. In certain implementations, the treatment method can pulsate the intensity of the light emitted from the light sources. The pulsating can be according to a duty cycle, percentage and/or a pattern, such as a sinusoidal, triangle function, step function, or saw tooth function. The intensity of individual light sources of the array or sub-arrays (i.e., groups) of light sources of the array can be pulsated or otherwise varied. The period and/or intensity of the pulsating can be varied according to the treatment methodology. In one example, the intensity of the light sources is pulsed at a 50% duty cycle at a rate of 2.5 Hz. In another example, the light sources are pulsed between on/off (100%) at a rate between 1-500 Hz. In another example, the light sources are pulsed at a 50% duty cycle at 10 Hz.

The arrays can sweep through various wavelengths over time. In certain implementations, the arrays of the wearable garment can include light sources with one or more wavelength or ranges of wavelengths. The treatment method can include sweeping through the wavelengths or ranges of wavelengths. This can be accomplished by coordinated powering up and down of individual light sources or sub-arrays of light sources (e.g., with corresponding wavelengths). The period and/or selection of wavelengths of the sweeping can be varied according to the treatment methodology. In one example, the array can include light sources that emit light at 630 nm, 680 nm, 810 nm, 830 nm and/or 904. The array can sweep through the wavelengths sequentially in either or both directions (descending or ascending wavelengths), randomly, or according to some other pattern.

### Wearable Apparatus for Treatment of Respiratory Disorders

The wearable garment for use as described herein may take a variety of forms, including but not limited to a strap, vest, shirt, jacket, sleeve, gown, or one or more pads. In one embodiment, one or more pads containing an array of light sources may be secured to a human body using one or more straps. The wearable garment provides freedom of movement such that the user is free to move about, and in some cases, engage in normal daily activities. In some embodiments, a portable power source is provided to enhance freedom of movement. In other embodiments, the wearable garment may be attached to a non-portable power source, but provide limited freedom of movement by having a tether to the power source. In some embodiments, the wearable garment permits phototherapy to be provided while the user is seated or standing.

In various embodiments, the wearable garment is configured to direct photon energy output to at least 20%, at least 50%, at least 75%, or 100% of the circumference around the torso. In various embodiments, the wearable garment is configured to direct at least 50%, at least 60%, at least 75%, at least 90%, or 100% of its photon energy output below the chest line of a human body 1 (i.e., below a line defined by the armpits).

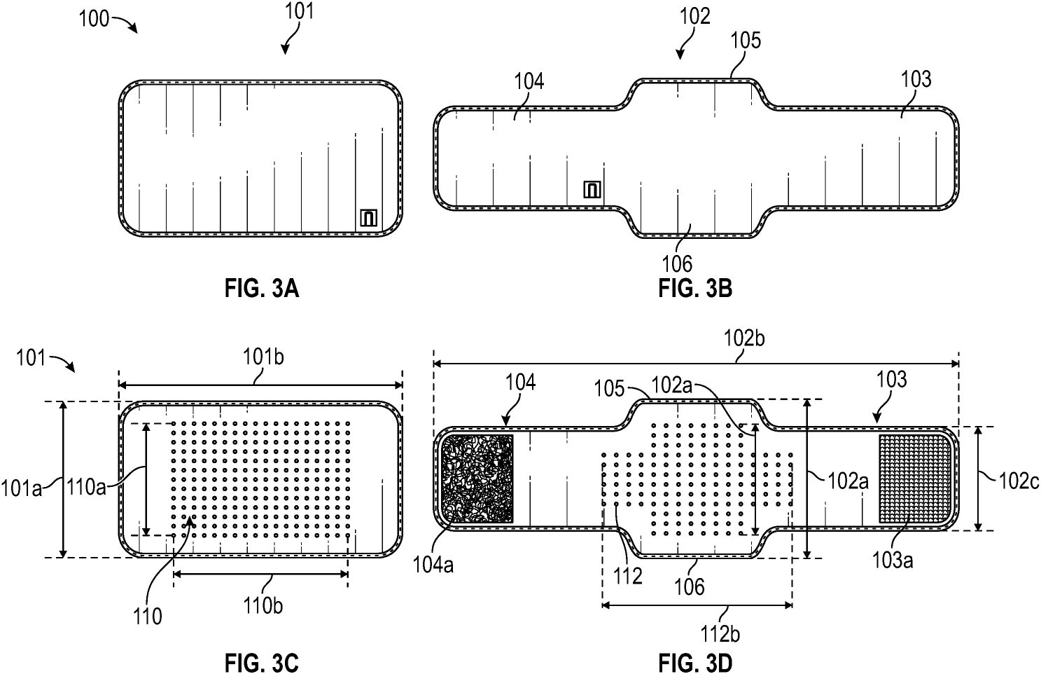

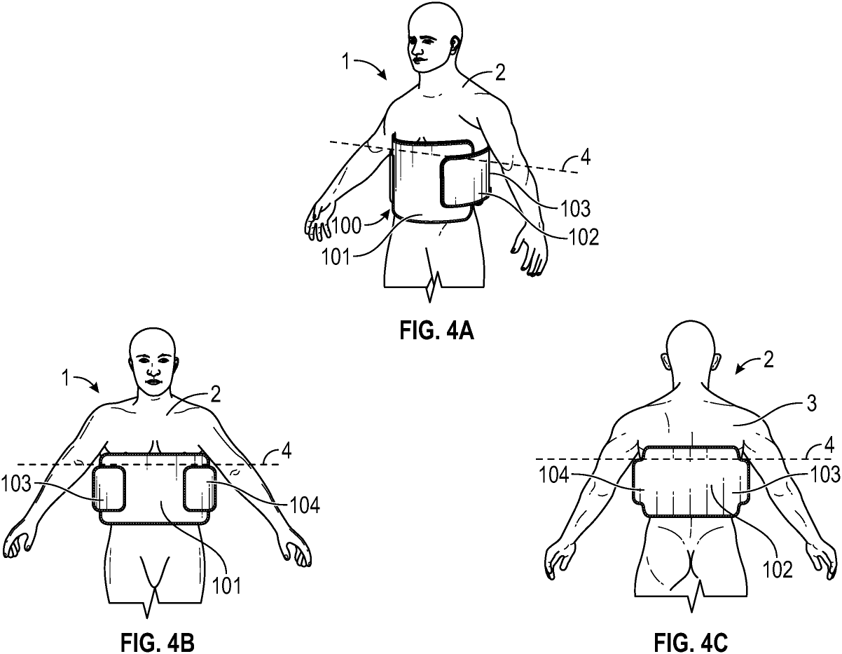

FIGS. 3A-4C show an embodiment of a wearable garment 100 for phototherapy of the torso region 2 of the human body 1. The wearable garment 100 can be formed of one or more layers of textile, polymer, and/or other natural or synthetic materials. In one example, the material can include a medical-grade silicone. For example, in one embodiment, the array of light sources are imbedded in a flexible sheet of medical-grade silicone. This sheet of medical-grade silicone can then be joined to or incorporated into other materials, such as fabric, to form the final wearable garment 100. The one or more layers can be flexible so as to be able to wrap the wearable garment 100 about the torso region 2. The wearable garment 100 can be long enough to wrap entirely or partially around the torso region 1 of the human body 2. The wearable garment 100 can form an enclosed loop. The enclosed loop can facilitate maintaining the wearable garment 100 in a desired position about the torso region 2.

The wearable garment 100 can include a two-piece construction including a front piece 101. The front piece 101 can be generally rectangularly shaped, although this is not required. The front piece 101 can include a width 101a and a length 101b. The width 101a and the length 101b can be selected to fully or partially cover the torso region 2 of the human body 1. In certain examples, the width 101a can be within a range of 4 in. to 18 in. In certain examples, the length 101b can be within a range of 4 in. to 36 in.

An inner side of the front piece 101 can include an array of light sources 110. The array 110 can include a plurality of light sources, including coherent and/or non-coherent light sources. The array 110 may include LEDs, filtered lamp light, and/or laser light. The array 110 can include a width 110a and a length 110b The light sources of the array 110 can be distributed evenly or unevenly along the width 110a and/or the length 110b. The width 110a and/or the length 110b of the array 110 can be within a range of 12 in. to 36 in. The array 110 can be spaced from the edges of the front piece 101. The array 110 can be spaced to be within 0 in. to 4 in. of the edges of the front piece 101. The light sources of the array 110 can be formed in a grid or other pattern. The array 110 can comprise sub-arrays. As described above, the sub-arrays may be independently controllable, and/or include light sources having all the same wavelength or different wavelengths. As discussed above, the light sources of the array 110 can include lenses that protrude outwardly from the inner side of the front piece 101 and facilitate contact with the skin of the human body 1.

The wearable garment 100 can include a back piece 102. The back piece 102 can include a central portion and one or more strap portions 103, 104 attached thereto or formed therewith. The central portion can include a width 102a. The central portion and the strap portions 103, 104 can include a length 102b. The length 102b can be selected to fully or partially cover the torso region 2 of the human body 1 (e.g., in combination with the front piece 101). In certain examples, the width 102a of the back piece 102 can be within a range of 4 in. to 18 in. In certain examples, the length 102b of the back piece 102 can be within a range of 12 in. to 52 in.

The strap portion 103 and/or the strap portion 104 can have a width 102c. The width 102a of the central portion can be greater than the width 102c of one or both the strap portions 103, 104. The width 102a of the central portion can define upper and/or lower wing portions 105, 106 that extend laterally relative to the width 102c of the strap portions 103, 104. In certain examples, the width 102c can be within a range of 1 in. to 12 in. An upper wing portion 105 can extend above a chest line 4. The chest line 4 can extend around a circumference of the torso region 2 at about the level of the armpits of the human body. The upper wing portion 105 can extend within a range of 0 in. to 6 in. above the chest line 4 when worn on the torso region 2.

An inner side of the back piece 102 can include an array of light sources 112. The array 112 can include a plurality of light sources, including coherent and/or non-coherent light sources. The array 112 may include LEDs, filtered lamp light, and/or laser light. The array 112 can include a width 112a and a length 112b The light sources of the array 112 can be distributed evenly or unevenly along the width 112a and/or the length 112b. The width 112a and/or the length 112b of the array 112 can be within a range of 12 in. to 36 in. The array 112 can be spaced from the edges of the back piece 102. The array 112 can be spaced to be within 0 in. to 4 in. of the edges of the back piece 102. The array 112 can extend onto the wing portions 105 and/or 106. The light sources of the array 112 can be formed in a cross-shaped grid, rectangular grid or other pattern. The light sources of the array 111 can be formed in a grid or other pattern. The array 112 can comprise sub-arrays. As described above, the sub-arrays may be independently controllable, and/or include light sources having all the same wavelength or different wavelengths. As discussed above, the light sources of the array 112 can include lenses that protrude outwardly from the inner side of the back piece 102 and facilitate contact with the skin of the human body 1.

The array 110 and/or the array 112 can extend about the circumference of the torso region (e.g., about the chest line 4). Depending on the lengths of the arrays, the photon energy output of the light sources can be directed to at least 20%, at least 50%, at least 75%, or 100% of the circumference around the torso.

The light sources of the arrays 110, 112 can include discrete lights that are connected with wires that enable the wearable garment 100 to retain flexibility for wrapping about the torso region. The wiring of the light sources can be embedded in between one or more layers of the wearable garment 100. The lenses of the light sources can extend through or couple with an exterior layer of the wearable garment 100.

The front piece 101 can couple with the straps 103, 104 of the back piece 102. The straps 103, 104 can include hook-and-loop portions 103a, 104a and an outer side of the front piece 101 can include corresponding hook-and-loop (e.g., VELCRO) material. In other implementations, other mechanical fasteners such clasps, belts, magnets, or other means can couple the straps 103, 104 on one or both side of the front piece 101. Accordingly, a circumference of the wearable garment 100 can be adjustable to fit a variety of torso regions 2.

The wearable garment 100 can include a power source, such as a battery or connect with a power source (e.g., AC/DC outlet) for providing power to the arrays of light sources. A battery is particularly promising in embodiments where the source of photonic energy comprises LEDs or other low energy consuming sources. The front piece 101 and the back piece 102 can be powered independently, or electrically connected, such as through a wire or wireless power connection. The wearable garment 100 can include a user interface, such as a switch, wireless connection (e.g., BLUETOOTH), or LCD screen. The user interface can allow powering on and off, and/or the selection of treatment regimes, such as times, intensities, pulsating patterns, sweeping patterns, and/or wavelengths or combinations thereof.

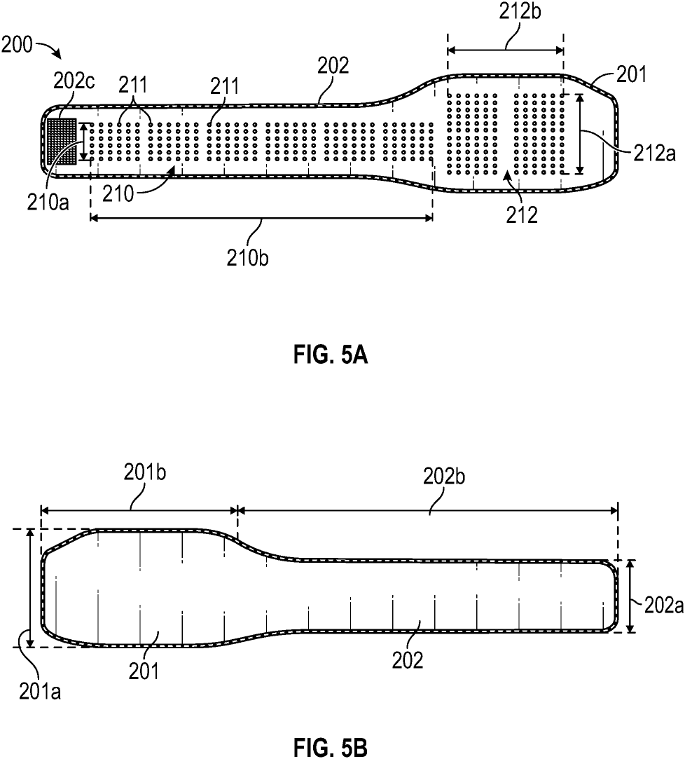

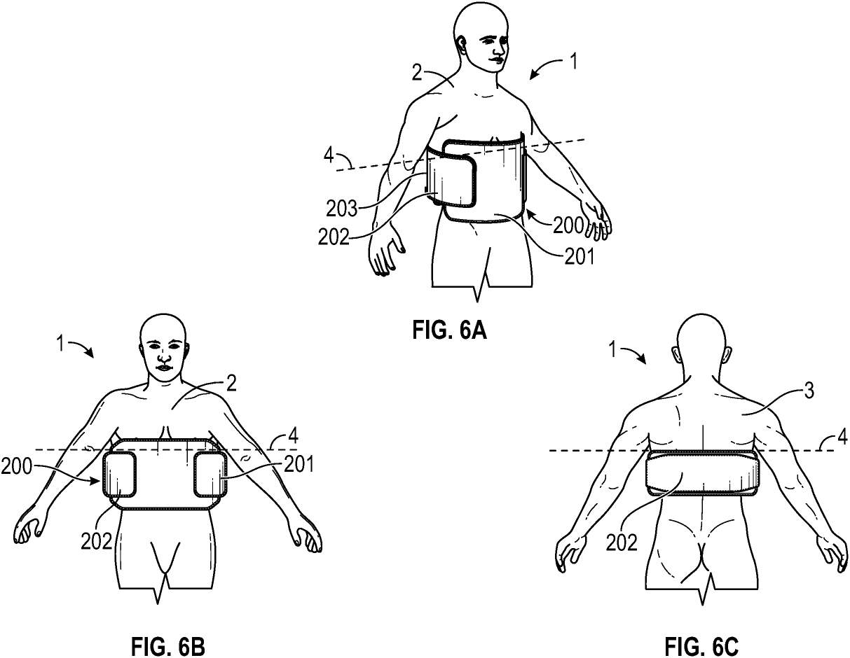

FIGS. 5A-6C show another embodiment of a wearable garment 200 for phototherapy of the torso region 2 of the human body 1. The wearable garment 200 can be formed of one or more layers of textile, polymer, silicone, and/or other natural or synthetic materials. The one or more layers can be flexible so as to be able to wrap the wearable garment 200 about the torso region 2. The wearable garment 200 can be long enough to wrap entirely or partially around the torso region 1 of the human body 2. The wearable garment 200 can form an enclosed loop. The enclosed loop can facilitate maintaining the wearable garment 200 in a desired position about the torso region 2.

The wearable garment 200 can include a single-piece construction including a first portion 201 and a second portion 202. The first portion 201 can be a strap portion. The first portion 201 can include a width 201a and a length 201b. The width 201a and the length 201b can be selected to fully or partially cover a back of the torso region 2 of the human body 1. In certain examples, the width 201a can be within a range of 4 in. to 18 in. In certain examples, the length 201b can be within a range of 4 in. to 36 in.

An inner side of the first portion 201 can include an array of light sources 210. The array 210 can include a plurality of light sources, including coherent and/or non-coherent light sources. The array 210 may include LEDs, filtered lamp light, and/or laser light. The array 210 can include a width 210a and a length 210b The light sources of the array 210 can be distributed evenly or unevenly along the width 210a and/or the length 210b. The width 210a of the array 210 can be within a range of 2 in. to 18 in. The length 210b of the array 210 can be within a range of 12 in. to 48 in. The array 210 can be spaced from the edges of the first portion 201. The array 210 can be spaced to be within 0 in. to 4 in. of the edges of the first portion 201. The light sources of the array 210 can be formed in a grid or other pattern. As shown, the array 210 can comprise sub-arrays 211. As described above, the sub-arrays 211 may be independently controllable, and/or include light sources having all the same wavelength or different wavelengths. As discussed above, the light sources of the array 210 can include lenses that protrude outwardly from the inner side of the first portion 201 and facilitate contact with the skin of the human body 1.

The wearable garment 200 can include a second portion 202. The second portion 202 can be designed to fit over a front of the torso region 2. The central portion can include a width 202a. The length 202b can be selected to cover or partially cover the torso region 2 of the human body 1 (e.g., in combination with the first portion 201). In certain examples, the width 202a of the second portion 202 can be within a range of 4 in. to 18 in. In certain examples, the length 202b of the second portion 202 can be within a range of 8 in. to 36 in.

The width 201a of the second portion 201 can be greater than the width 202a of the first portion 202. The width 201a can define upper and/or lower wing portions that extend laterally relative to the width 202a. In An upper wing portion can extend above a chest line 4. The chest line 4 can extend around a circumference of the torso region 2 at about the level of the armpits of the human body. The upper wing portion can extend within a range of 0 in. to 6 in. above the chest line 4 when worn on the torso region 2.

An inner side of the second portion 202 can include an array of light sources 212. The array 212 can include a plurality of light sources, including coherent and/or non-coherent light sources. The array 212 may include LEDs, filtered lamp light, and/or laser light. The array 212 can include a width 212a and a length 212b The light sources of the array 212 can be distributed evenly or unevenly along the width 212a and/or the length 212b. The width 212a and/or the length 212b of the array 212 can be within a range of 12 in. to 36 in. The array 212 can be spaced from the edges of the second portion 202. The array 212 can be spaced to be within 0 in. to 4 in. of the edges of the second portion 202. The array 212 can extend onto the wing portions 105 and/or 106. The light sources of the array 212 can be formed in a cross-shaped grid, rectangular grid or other pattern. The array 212 can include sub-arrays 213. As discussed above, the light sources of the array 212 can include lenses that protrude outwardly from the inner side of the second portion 202 and facilitate contact with the skin of the human body 1.

The array 210 and/or the array 212 can extend about the circumference of the torso region (e.g., about the chest line 4). Depending on the lengths of the arrays, the photon energy output of the light sources can be directed to at least 20%, at least 50%, at least 75%, or 100% of the circumference around the torso.

The light sources of the arrays can include discrete lights that are connected with wires that enable the wearable garment 200 to retain flexibility for wrapping about the torso region. The wiring of the light sources can be embedded in between one or more layers of the wearable garment 200. The lenses of the light sources can extend through or couple with an exterior layer of the wearable garment 200.

The first portion 201 can couple with the second portion 202 to form the closed loop. The first portion 202 can include hook-and-loop portions 202c and an outer side of the second portion 202 can include corresponding hook-and-loop material. In other implementations, other mechanical fasteners such clasps, belts, magnets, or other means can couple the first and second portions 201, 202. Accordingly, a circumference of the wearable garment 200 can be adjustable to fit a variety of torso regions 2.

The wearable garment 200 can include a power source, such as a battery or connect with a power source for providing power to the arrays of light sources. The arrays of the first portion 201 and the second portion 202 can be powered independently, or electrically connected, such as through a wire or wireless power connection. The wearable garment 200 can include a user interface, such as a switch, wireless connection (e.g., BLUETOOTH), or LCD screen. The user interface can allow powering on and off, and/or the selection of treatment regimes, such as times, intensities, pulsating patterns, sweeping patterns, and/or wavelengths or combinations thereof.

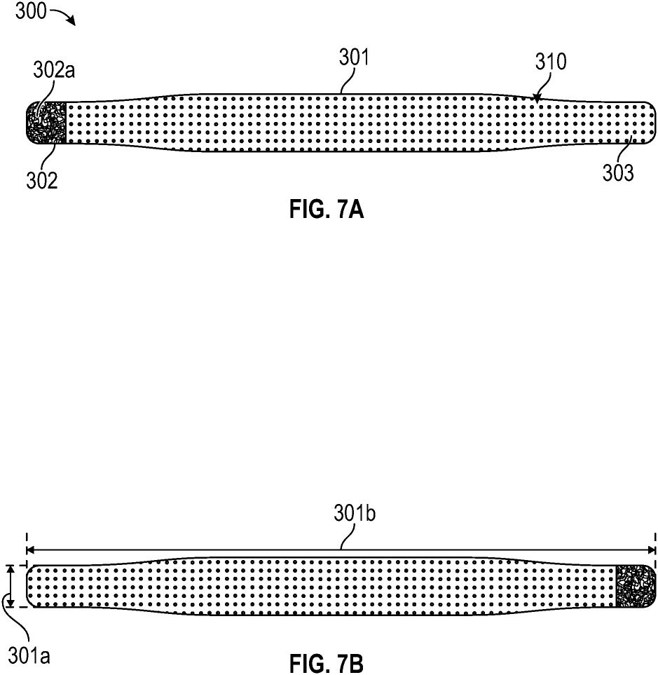

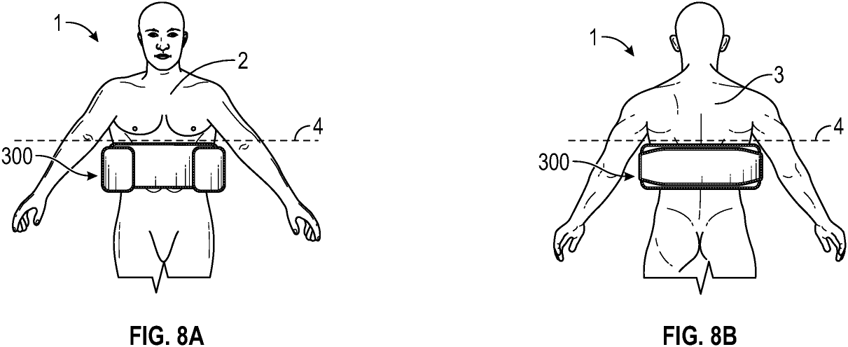

FIGS. 7A-8B show another embodiment of a wearable garment 300 for phototherapy of the torso region 2 of the human body 1. The wearable garment 300 can be formed of one or more layers of textile, polymer, silicone, and/or other natural or synthetic materials. The one or more layers can be flexible so as to be able to wrap the wearable garment 300 about the torso region 2. The wearable garment 300 can be long enough to wrap entirely or partially around the torso region 1 of the human body 2. The wearable garment 300 can form an enclosed loop. The enclosed loop can facilitate maintaining the wearable garment 300 in a desired position about the torso region 2.

The wearable garment 300 can include a single-piece construction including a first portion 301 and straps 302, 303. The first portion 301 can be a central portion. The wearable garment 300 can include a width 301a and a length 301b. The width 301a and the length 301b can be selected to fully or partially cover a back of the torso region 2 of the human body 1. In certain examples, the width 301a can be within a range of 2 in. to 18 in. In certain examples, the length 301b can be within a range of 24 in. to 52 in.

An inner side of the first portion 301 can include an array of light sources 310. The array 310 can include a plurality of light sources, including coherent and/or non-coherent light sources. The array 310 may include LEDs, filtered lamp light, and/or laser light. The light sources of the array 310 can be distributed evenly or unevenly along the width 301a and/or the length 301b. The array 310 can be spaced from the edges of the first portion 301. The array 310 can be spaced to be within 0 in. to 4 in. of the edges of the first portion 301. The light sources of the array 310 can be formed in a grid or other pattern. The array 310 can extend on the straps 302 and/or 303. The array 310 can comprise sub-arrays. As described above, the sub-arrays may be independently controllable, and/or include light sources having all the same wavelength or different wavelengths. As discussed above, the light sources of the array 310 can include lenses that protrude outwardly from the inner side of the first portion 301 and facilitate contact with the skin of the human body 1.

The wearable garment 300 can include a straps 302, 303. The straps 302, 303 can be designed to fit over a front of the torso region 2. The width of the first portion 301 can be greater than the width straps 302, 303. The first portion 301 can extend to a chest line 4. The chest line 4 can extend around a circumference of the torso region 2 at about the level of the armpits of the human body.

The array 310 can extend about the circumference of the torso region (e.g., about the chest line 4). Depending on the lengths of the arrays, the photon energy output of the light sources can be directed to at least 30%, at least 50%, at least 75%, or 100% of the circumference around the torso.

The light sources of the array 310 can include discrete lights that are connected with wires that enable the wearable garment 300 to retain flexibility for wrapping about the torso region. The wiring of the light sources can be embedded in between one or more layers of the wearable garment 300. The lenses of the light sources can extend through or couple with an exterior layer of the wearable garment 300.

The straps 302, 303 can couple to form the closed loop. The strap 302 can include hook-and-loop portions 302a and an outer side of the straps 303 can include corresponding hook-and-loop material. In other implementations, other mechanical fasteners such clasps, belts, magnets, or other means can couple the first and second straps 302, 303. Accordingly, a circumference of the wearable garment 300 can be adjustable to fit a variety of torso regions 2.

The wearable garment 300 can include a power source, such as a battery or connect with a power source for providing power to the arrays of light sources. The array of the first portion 301 can be powered independently, or electrically connected, such as through a wire or wireless power connection. The wearable garment 300 can include a user interface, such as a switch, wireless connection (e.g., BLUETOOTH), or LCD screen. The user interface can allow powering on and off, and/or the selection of treatment regimes, such as times, intensities, pulsating patterns, sweeping patterns, and/or wavelengths or combinations thereof.

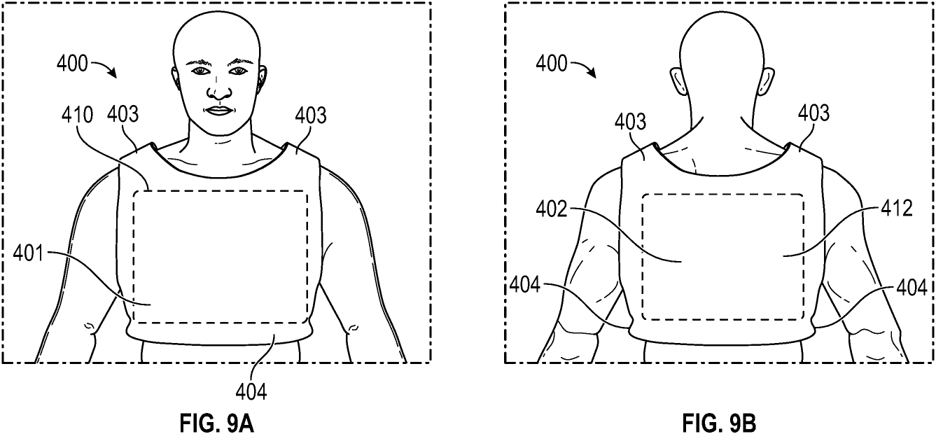

FIGS. 9A-B show an embodiment of a wearable garment 400 for phototherapy of the torso region 2 of the human body 1 in the form of a vest. The wearable garment 400 can be formed of one or more layers of textile, silicone, polymer, and/or other natural or synthetic materials. The one or more layers can be flexible so as to be able to wrap the wearable garment 400 about the torso region 2.

The wearable garment 400 can include a front piece 401 and a back piece 402. The front piece 401 can be connected with the back piece 402 by one or more straps 403 and/or 404. The straps 403, 404 can extend over the shoulders and/or under the arms of the human body 1. The straps 403, 404 can be adjustable to position the wearable garment about the torso region 2.

The front piece 401 can fully or partially cover the front of the torso region 2 of the human body 1. An inner side of the front piece 401 can include an array of light sources 410. The array 410 can include a plurality of light sources, including coherent and/or non-coherent light sources. The array 410 may include LEDs, filtered lamp light, and/or laser light. The light sources of the array 410 can be distributed evenly or unevenly along the front piece 401. The array 410 can be within a range of 12 in. to 36 in. The array 410 can be spaced from the edges of the front piece 401. The array 410 can be spaced to be within 0 in. to 4 in. of the edges of the front piece 401. The light sources of the array 410 can be formed in a grid or other pattern. The array 410 can comprise sub-arrays. As described above, the sub-arrays may be independently controllable, and/or include light sources having all the same wavelength or different wavelengths. As discussed above, the light sources of the array 410 can include lenses that protrude outwardly from the inner side of the front piece 401 and facilitate contact with the skin of the human body 1.

The back piece 402 can fully or partially cover the back of the torso region 2 of the human body 1. An inner side of the back piece 402 can include an array of light sources 412. The array 412 can include a plurality of light sources, including coherent and/or non-coherent light sources. The array 412 may include LEDs, filtered lamp light, and/or laser light. The array 412 can include a width 412a and a length 412b. The light sources of the array 412 can be distributed evenly or unevenly along the back piece 402. The array 412 can be spaced from the edges of the back piece 402. The array 412 can be spaced to be within 0 in. to 4 in. of the edges of the back piece 402. The array 412 can extend onto the wing portions 405 and/or 406. The light sources of the array 412 can be formed in a cross-shaped grid, rectangular grid or other pattern. The light sources of the array 412 can be formed in a grid or other pattern. The array 412 can comprise sub-arrays. As described above, the sub-arrays may be independently controllable, and/or include light sources having all the same wavelength or different wavelengths. As discussed above, the light sources of the array 412 can include lenses that protrude outwardly from the inner side of the back piece 402 and facilitate contact with the skin of the human body 1.

The array 410 and/or the array 412 can extend about the circumference of the torso region (e.g., about the chest line 4). Depending on the lengths of the arrays, the photon energy output of the light sources can be directed to at least 20%, at least 50%, at least 75%, or 100% of the circumference around the torso.

The light sources of the arrays 440, 412 can include discrete lights that are connected with wires that enable the wearable garment 400 to retain flexibility for wrapping about the torso region. The wiring of the light sources can be embedded in between one or more layers of the wearable garment 400. The lenses of the light sources can extend through or couple with an exterior layer of the wearable garment 400.

The wearable garment 400 can include a power source, such as a battery or connect with a power source for providing power to the arrays of light sources. The front piece 401 and the back piece 402 can be powered independently, or electrically connected, such as through a wire or wireless power connection. The wearable garment 400 can include a user interface, such as a switch, wireless connection (e.g., BLUETOOTH), or LCD screen. The user interface can allow powering on and off, and/or the selection of treatment regimes, such as times, intensities, pulsating patterns, sweeping patterns, and/or wavelengths or combinations thereof.

### Certain Terminology

Terms of orientation used herein, such as “top,” “bottom,” “proximal,” “distal,” “longitudinal,” “lateral,” and “end,” are used in the context of the illustrated example. However, the present disclosure should not be limited to the illustrated orientation. Indeed, other orientations are possible and are within the scope of this disclosure. Terms relating to circular shapes as used herein, such as diameter or radius, should be understood not to require perfect circular structures, but rather should be applied to any suitable structure with a cross-sectional region that can be measured from side-to-side. Terms relating to shapes generally, such as “circular,” “cylindrical,” “semi-circular,” or “semi-cylindrical” or any related or similar terms, are not required to conform strictly to the mathematical definitions of circles or cylinders or other structures, but can encompass structures that are reasonably close approximations.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

The terms “approximately,” “about,” and “substantially” as used herein represent an amount close to the stated amount that still performs a desired function or achieves a desired result. For example, in some examples, as the context may dictate, the terms “approximately,” “about,” and “substantially,” may refer to an amount that is within less than or equal to 10% of the stated amount. The term “generally” as used herein represents a value, amount, or characteristic that predominantly includes or tends toward a particular value, amount, or characteristic. As an example, in certain examples, as the context may dictate, the term “generally parallel” can refer to something that departs from exactly parallel by less than or equal to 20 degrees. All ranges are inclusive of endpoints.

### Summary

Several illustrative examples of treatment methods and apparatuses have been disclosed. Although this disclosure has been described in terms of certain illustrative examples and uses, other examples and other uses, including examples and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various examples. All possible combinations and subcombinations of elements and components described herein are intended to be included in this disclosure. No single feature or group of features is necessary or indispensable.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, structures, and/or devices disclosed or illustrated in one example in this disclosure can be combined or used with (or instead of) any other portion of any of the steps, processes, structures, and/or devices disclosed or illustrated in a different example or flowchart. The examples described herein are not intended to be discrete and separate from each other. Combinations, variations, and some implementations of the disclosed features are within the scope of this disclosure.

While operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Additionally, the operations may be rearranged or reordered in some implementations. Also, the separation of various components in the implementations described above should not be understood as requiring such separation in all implementations, and it should be understood that the described components and systems can generally be integrated together in a single product or packaged into multiple products. Additionally, some implementations are within the scope of this disclosure.

Further, while illustrative examples have been described, any examples having equivalent elements, modifications, omissions, and/or combinations are also within the scope of this disclosure. Moreover, although certain aspects, advantages, and novel features are described herein, not necessarily all such advantages may be achieved in accordance with any particular example. For example, some examples within the scope of this disclosure achieve one advantage, or a group of advantages, as taught herein without necessarily achieving other advantages taught or suggested herein. Further, some examples may achieve different advantages than those taught or suggested herein.

Some examples have been described in connection with the accompanying drawings. The figures are drawn and/or shown to scale, but such scale should not be limiting, since dimensions and proportions other than what are shown are contemplated and are within the scope of the disclosed invention. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, any methods described herein may be practiced using any device suitable for performing the recited steps.

For purposes of summarizing the disclosure, certain aspects, advantages and features of the inventions have been described herein. Not all, or any such advantages are necessarily achieved in accordance with any particular example of the inventions disclosed herein. No aspects of this disclosure are essential or indispensable. In many examples, the devices, systems, and methods may be configured differently than illustrated in the figures or description herein. For example, various functionalities provided by the illustrated modules can be combined, rearranged, added, or deleted. In some implementations, additional or different processors or modules may perform some or all of the functionalities described with reference to the examples described and illustrated in the figures. Many implementation variations are possible. Any of the features, structures, steps, or processes disclosed in this specification can be included in any example.

In summary, various examples of treatment methods and apparatuses and related methods have been disclosed. This disclosure extends beyond the specifically disclosed examples to other alternative examples and/or other uses of the examples, as well as to certain modifications and equivalents thereof. Moreover, this disclosure expressly contemplates that various features and aspects of the disclosed examples can be combined with, or substituted for, one another. Accordingly, the scope of this disclosure should not be limited by the particular disclosed examples described above, but should be determined only by a fair reading of the claims.

What is claimed is:

1. A method of treating pulmonary inflammation using a wearable garment, comprising:
placing a wearable garment in contact with a torso of a human body having the pulmonary inflammation, the wearable garment including an irradiation source having a photon energy output primarily emitted in a spectral wavelength range of between 600-1600 nm; and
directing the photon energy output of the wearable garment primarily to the torso of the human body.

2. The method according to claim 1, further comprising directing the photon energy output of the wearable garment primarily to the torso of the human body for a treatment period of between 10 and 40 minutes.

3. The method according to claim 1, further comprising directing the photon energy output of the wearable garment primarily to the torso of the human body for a treatment period of at least 10 minutes.

4. The method according to claim 1, wherein the photon energy output is emitted primarily in a spectral wavelength range between 800-1000 nm.

5. The method according to claim 1, wherein the photon energy output is emitted primarily in a spectral wavelength range between 830-980 nm.

6. The method according to claim 1, wherein the photon energy output is emitted primarily in a spectral wavelength range between 630-650 nm.

7. The method according to claim 1, wherein the photon energy output is emitted primarily at a spectral wavelength of about 630 nm.

8. The method according to claim 1, wherein the photon energy output is emitted primarily at a spectral wavelength of about 680 nm.

9. The method according to claim 1, wherein the photon energy output is emitted primarily at a spectral wavelength of about 810 nm.

10. The method according to claim 1, wherein the photon energy output is emitted primarily at a spectral wavelength of about 830 nm.

11. The method according to claim 1, wherein the photon energy output is emitted primarily at a spectral wavelength of about 904 nm.

12. The method according to claim 1, wherein the irradiation source comprises a coherent light sources.

13. The method according to claim 12, wherein the coherent light sources is a laser.

14. The method according to claim 1, wherein the irradiation source comprises a non-coherent light sources.

15. The method according to claim 14, wherein the non-coherent light sources is a light emitting diodes (LED).

16. The method according to claim 14, wherein the non-coherent light sources is a filtered lamp.

17. The method according to claim 1, wherein the irradiation source is one of an array of irradiation sources on the wearable garment.

18. The method according to claim 1, wherein the photon energy output is emitted in a pulsating pattern.

19. The method according to claim 1, wherein the photon energy output emitted sweeps over a range of spectral wavelengths.

20. The method according to claim 19, wherein the range of spectral wavelengths is between 800-1000 nm.

21. The method according to claim 1, wherein the wearable garment comprises an adjustable strap and a front piece including a first array of irradiation sources configured to primarily direct the photon energy output from the first array toward a chest region of the human body, and wherein the strap is positioned circumferentially around the torso of the human body.

22. The method according to claim 21, wherein the adjustable strap further includes a second array of irradiation sources configured to primarily direct the photon energy output from the second array toward the chest region of the human body.

23. The method according to claim 21, wherein the wearable garment further includes a back piece including a second array of irradiation sources configured to primarily direct the photon energy output from the second array toward a back region of the human body opposite the chest region.

24. The method according to claim 1, wherein the wearable garment comprises a vest including a front piece and a back piece configured to primarily direct the photon energy output to the torso of the human body.

25. The method according to claim 1, wherein the irradiation source includes a lens configured to protrude from an inner surface of the wearable garment such that the lens contacts an outer layer of skin of the torso of the human body.

26. The method according to claim 1, wherein the pulmonary inflammation is caused by a viral infection.

27. The method according to claim 26, wherein the viral infection is COVID-19.

28. The method according to claim 1, comprising directing the photon energy output of the wearable garment primarily to a chest region, a back region opposite a chest region, or both a chest region and a back region opposite a chest region of the human body.

29. The method according to claim 1, wherein the wearable garment is placed in contact with skin of the human body.

30. The method according to claim 1, wherein the photon energy output is directed to at least 20% of a circumference around the torso.

31. The method according to claim 1, wherein the photon energy output is directed to at least 50% of a circumference around the torso.

32. The method according to claim 1, wherein the photon energy output is directed to at least 75% of a circumference around the torso.

33. The method according to claim 1, wherein the photon energy output is directed to 100% of a circumference around the torso.

34. An apparatus for treatment of COVID-19 respiratory virus, comprising:
a wearable garment configured to at least partially cover a chest region of a human body, the wearable garment including:
a front piece having a first array of irradiation sources;
wherein the first array of irradiation sources are configured to direct a photon energy output primarily in a spectral wavelength range between 600-1600 nm at the chest region when the wearable garment is being worn.

35. The treatment apparatus of claim 34, wherein the first array of irradiation sources comprises LEDs.

36. The treatment apparatus of claim 34, further comprising a battery configured to power the first array of irradiation sources.

37. The treatment apparatus of claim 34, wherein the photon energy output is emitted primarily in a spectral wavelength range between 800-1000 nm.

38. The treatment apparatus of claim 34, wherein the photon energy output is emitted primarily in a spectral wavelength range between 830-980 nm.

39. The treatment apparatus of claim 34, wherein the photon energy output is emitted primarily in a spectral wavelength range between 630-650 nm.

40. The treatment apparatus of claim 34, wherein the photon energy output is emitted primarily at a spectral wavelength of about 830 nm.

41. The treatment apparatus of claim 34, wherein the photon energy output is emitted primarily at a spectral wavelength of about 904 nm.

42. The treatment apparatus of claim 34, wherein first array of irradiation sources comprises coherent light sources.

43. The treatment apparatus of claim 34, wherein the first array of irradiation sources is configured to emit photon energy in a pulsating pattern.

44. The treatment apparatus of claim 34, wherein the first array of irradiation sources is configured to emit photon energy in a sweep over a range of spectral wavelengths.

45. The treatment apparatus of claim 44, wherein the range of spectral wavelengths is between 800-1000 nm.

46. The treatment apparatus of claim 34, wherein the wearable garment further comprises:
an adjustable strap;
a mechanical fastener to couple the front piece with the chest region of the human body; and
a second array of irradiation sources configured to primarily direct the photon energy output in the chest region of the human body.

47. The treatment apparatus of claim 46, wherein the second array of irradiation sources is on the adjustable strap.

48. The treatment apparatus of claim 46, wherein the adjustable strap includes a widened portion forming a back piece, the back piece including the second array of irradiation sources.

49. The treatment apparatus of claim 34, wherein the front piece of the wearable garment has a width that extends above a chest line of the human body.

50. The treatment apparatus of claim 34, wherein the wearable garment comprises a vest including the front piece and a back piece.

51. The treatment apparatus of claim 34, wherein the irradiation source of the first array of irradiation sources include a lenses configured to protrude from an inner surface of the wearable garment such that the lenses are configured to contact an outer layer of skin of the chest region of the human body.
